(19) United States
(12) Patent Application Publication
(10) Pub. No.: US 2011/0196227 A1
(43) Pub. Date: Aug. 11, 2011

(54) Apparatus with a Combination of a Magnetic Resonance Apparatus and a Radiotherapy Apparatus

(76) Inventors: Patrick Gross, Langensendelbach (DE); Björn Heismann, Erlangen (DE)

(21) Appl. No.: 13/024,548

(22) Filed: Feb. 10, 2011

(30) Foreign Application Priority Data
Feb. 10, 2010 (DE) 10 2010 001 746.9

(51) Int. Cl. A61B 5/055; A61N 5/00

(52) U.S. Cl. 600/411

(57) ABSTRACT

An apparatus with a combination of a magnetic resonance apparatus is proposed. The apparatus has at least one main magnet for generating a magnetic field in an examination area for a magnetic resonance measurement and a radiotherapy apparatus which is designed for generating a particle beam. A direction of a speed of the particle beam is aligned substantially in parallel to a direction of a magnetic flux density of the magnetic field.

### CROSS REFERENCE TO RELATED APPLICATIONS

This application claims priority of German application No. 10 2010 001 746.9 filed Feb. 10, 2010, which is incorporated by reference herein in its entirety.

### FIELD OF THE INVENTION

The present invention relates to an apparatus with a combination of a magnetic resonance apparatus, having at least one main magnet for generating a magnetic field in an examination area for a magnetic resonance measurement and a radiotherapy apparatus designed for generating a particle beam.

### BACKGROUND OF THE INVENTION

Radiotherapy generally involves the irradiation of a target within a human body in order to treat an illness, especially a tumor, for example. In such cases a high dose of radiation is explicitly introduced into an irradiation region (isocenter) of the human body by a radiotherapy apparatus. During radiation treatment the problem that often arises is that of the movement or displacement of the irradiation region. Thus for example a tumor in a stomach area moves during the breathing process of a patient. On the other hand a tumor can have increased in size or also reduced in size within the period between irradiation planning and the actual irradiation.

It has been proposed that a location of an irradiation target in a patient be checked during irradiation by medical imaging. This makes it possible to control a beam and/or a beam guidance for the irradiation or to abort the irradiation if necessary. In addition adjusting the beam focus in respect of the actual position of the irradiation region is of great interest.

In particular a combination of the radiotherapy apparatus with a magnetic resonance apparatus is especially advantageous. By comparison with a computed tomography apparatus for example, this exhibits a high soft tissue resolution so that an advantageous contrast can be presented in this area.

For efficient irradiation an irradiation source of the radiotherapy apparatus is positioned as close as possible to a patient. For this purpose the irradiation source is arranged at least partly within the magnetic resonance apparatus and especially within a magnetic field of the magnetic resonance apparatus. However this arrangement has the disadvantage of an electron path of electrons of an electron beam of the radiotherapy device being disrupted by the magnetic field of the magnetic resonance apparatus.

WO 03/008986 A2 proposes a division of gradient coils of the magnetic resonance apparatus and an adapted design of a main magnet, so that an almost magnetic-field-free space is created outside the magnetic resonance apparatus. However the disadvantage of this arrangement is that the apparatus extends over a large area and in addition only one irradiation angle is available for radiation treatment, In addition the divided gradient coils lead to obvious disadvantages in the image quality of medical imaging. Furthermore a beam of the radiotherapy apparatus must penetrate the steel body of the magnet, leading to a deterioration and/or degradation of the beam profile and the beam intensity.

Also known from U.S. Pat. No. 6,198,957 B1 is a combination of a magnetic resonance apparatus and a radiotherapy apparatus in which however an X-ray beam and/or gamma beam is generated for irradiation outside the magnetic resonance apparatus and thus outside an effective range of a magnetic field. The X-ray beam is generated at a very great distance from the actual treatment area using this method, so that the apparatus also extends over a large area, especially if an angle of irradiation is varied. In addition, because of the large distance, a high dose of radiation must be generated in order to achieve the required depth of penetration of the radiation for the radiation treatment.

Also known from DE 102008007245 A1 is guidance of an electron beam along the main axis of the magnetic resonance apparatus. The electron beam is diverted by 90° for a collision with a target. The electron beam and the target are arranged within a patient accommodation area of the magnetic resonance apparatus. However this means that the space available for the patient within the magnetic resonance apparatus is additionally restricted by the radiotherapy apparatus.

### SUMMARY OF THE INVENTION

The particular object underlying the present invention is to provide an apparatus with a combination of a magnetic resonance apparatus and a radiotherapy apparatus which provides a compact structure and high quality image monitoring by magnetic resonance measurement during radiotherapy. The object is achieved by the features of the independent claims. Further advantageous embodiments are described in the dependent claims.

The starting point for the invention is an apparatus with a combination of a magnetic resonance apparatus having at least one main magnet for generation of a magnetic field in an examination space for a magnetic resonance measurement and a radiotherapy apparatus which is provided for generating a particle beam.

It is proposed that a direction of a speed of the particle beam is aligned substantially in parallel to a direction of a magnetic flux density of the magnetic field. In this context a main magnet should especially be understood as a magnet which is designed to apply and/or to generate a constant and especially homogeneous magnetic field. The main magnet is preferably formed by a superconducting magnet. Furthermore an examination space is to be understood especially as a space and/or area of the magnetic resonance apparatus provided to accommodate a patient and in which an imaging examination and/or measurement can be carried out on a patient by means of the magnetic resonance apparatus and an irradiation by means of the radiotherapy apparatus. A radiotherapy apparatus is especially to be understood as an apparatus intended for the irradiation of an area, for example a tumor area of a patient, with an ionizing, high-energy radiation, wherein the ionizing, high-energy radiation acts on the patient from the outside. The ionizing, high-energy radiation is predominantly formed by gamma radiation and/or X-radiation and/or electron radiation, wherein the energy and/or dose power of the radiation is tailored to a type of tissue and/or on a position of the treatment area within the patient and especially below a patient's skin. Irradiation with neutrons and/or protons and/or heavy ions by means of radiotherapy is also conceivable. Furthermore a particle beam is to be understood in this context especially as a directed movement of a plurality of particles along a uniform direction of advance, such as a stream of particles for example with a substantially uniform direction of flight. The particle beam is especially formed by an electron beam which is focused on a treatment area of a patient and/or which is directed for creating an X-ray and/or gamma ray onto a target.

The greater the magnetic flux density of the magnetic field and/or the greater a distance to be covered by the particle beam within the magnetic field is embodied, the more strictly in this case are the criteria for a parallelism of the speed of the particle beam and the direction of the magnetic flux density of the magnetic field to be adhered to in order to prevent an undesired beam deflection outside a tolerance area. A change in the direction and/or orientation of the speed \vec{v} of the particle beam is made by a force \vec{F} which substantially depends on a vector product of the speedy \vec{v} of the particle beam and on the magnetic flux density \vec{B}₀, of the magnetic field: \vec{F}∝(\vec{v}×\vec{B}₀). Thus no force acts on particles of the particle beam which are moving at the speed \vec{v} which is aligned in parallel to the magnetic field \vec{B}₀, so that in this case an undesired adverse effect on the orientation and/or direction of the particle beam by the magnetic field can be prevented. Furthermore an undesired disruption of an imaging measurement which could be caused by a disruption in a homogeneity of the magnetic field by the particle beam is reduced and/or prevented, so that a high-quality image monitoring by a magnetic resonance measurement during a radiotherapy can be achieved. In addition the magnetic resonance apparatus can be employed with a high-field magnetic field since adverse effects caused by the magnetic resonance apparatus and radiotherapy apparatus are suppressed. In addition a compact layout of the apparatus can advantageously be achieved in that the beam path of the particle beam can be arranged in an especially space-saving manner at least partly within the magnetic resonance apparatus.

Preferably the particle beam runs at least partly within the magnetic resonance apparatus and especially preferably within an area of the magnetic resonance apparatus through which the magnetic field passes, so that an especially compact arrangement can be achieved.

It is further proposed that the particle beam runs substantially undiverted and especially undiverted within the magnetic field. This enables additional components for changing the direction of the particle beam to be advantageously dispensed with and thereby an especially compact apparatus to be achieved. In addition an undesired adverse effect of a homogeneity of the magnetic field caused by a change in direction of the particle beam can be advantageously prevented and thus an advantageous high quality of the imaging achieved by means of the magnetic resonance apparatus. A restriction of a magnetic field size, as is a consequence of a change in direction and/or a redirection of the particle beam, can be prevented in this case so that a high-field magnetic field can be used for a magnetic resonance measurement.

An undesired redirection of the particle beam, especially within the magnetic resonance apparatus, and thereby an adverse effect on a homogeneity of the magnetic field can be prevented if the particle beam is directed onto a treatment area before entry into an area of the magnetic resonance apparatus penetrated by the magnetic field. A treatment area in this case should especially be understood as a target area and/or an isocenter in which treatment is to be carried out by means of the radiotherapy apparatus and which preferably is located within a patient.

Furthermore it is proposed that the direction of the magnetic flux density of the magnetic field is substantially perpendicular to an alignment of a head-foot axis of a patient located in the examination area. The patient and especially the treatment area within the examination space can be made accessible in a constructively simple manner to irradiation by means of the radiotherapy apparatus. In this context a head-foot axis the patient should especially be understood as a longitudinal axis and/or a longitudinal extent of a patient located in the examination space and/or in an accommodation area of the magnetic resonance apparatus.

In an advantageous development of the invention it is proposed that the radiotherapy apparatus has at least one linear accelerator unit which is arranged at least partly within the magnetic resonance apparatus and especially advantageously at least partly within the area penetrated by the magnetic field. In this way an especially space-saving accelerator of the particles up to an end energy required for the irradiation is achieved.

In this context a linear accelerator unit (Linac) is especially to be understood as a unit for accelerating electrically-charged particles, especially electrons, wherein the particles are accelerated on a straight path. The particle beam is accelerated in this case by electrical alternating fields in a cylindrical hollow conductor. The hollow conductor is preferably arranged in a vacuum tube in this case so that undesired collations of the beam particles with air molecules and/or air particles are prevented. For radiotherapy electrons can typically be accelerated to energies up to an order of magnitude of several MeV. In addition it is also possible for the radiotherapy apparatus to feature an alternate accelerator unit to a linear accelerator unit.

Furthermore it is proposed that the radiotherapy apparatus has at least one target for generating a gamma and/or X-ray beam and that the target is arranged at least partly within the magnetic resonance apparatus. An especially space-saving arrangement and/or positioning of the target in the vicinity of the treatment region and thus especially close to the patient can be achieved. In addition this enables a beam focus, especially a point-type beam focus, to be explicitly focused on the treatment area and an undesired beam spread can be suppressed because of the short distance from the target to the treatment area. Furthermore a high radiation dose can act on the treatment area because of the short distance between the target and the treatment area. This can be achieved especially advantageously if the target is arranged within an area penetrated by the magnetic field. Preferably in this case the target is formed from a magnetic resonance-compatible, especially from a non-magnetizable material. A gamma beam should especially be understood here as a photon beam with especially ultra hard X-ray radiation, with the photons having a higher energy than an energy of the photons of the X-ray beam. An advantageous penetration depth can be achieved in this case during radiation treatment of a patient by means of the radiotherapy apparatus, so that especially tumors lying deeper can also be treated and/or irradiated by means of the radiotherapy apparatus. An energy of the photons can in such cases be tailored to a desired depth of penetration and/or a position of the treatment area within the patient. The greater the energy of the photons, the greater is also the depth of penetration of the photons into the body of the patient.

Furthermore it is proposed that the target be formed at least partly by a transmission target, enabling high-energy photons to be generated in a constructively simple manner, wherein the high-energy photons leave the target in a preferred direction which is aligned substantially in parallel to the direction of the speed of the particle beam. At the transmission target, the electrons of the electron beam of the linear accelerator unit striking the transmission target are decelerated and emit a braking radiation during this process which is formed by the high-energy photons. Basically, in an alternate embodiment of the invention, the target can also be formed by a reflection target.

It is further proposed that the radiotherapy apparatus features at least one collimator which is arranged at least partly within the magnetic resonance apparatus (2). Preferably the collimator is provided for a substantially parallel alignment of gamma and/or X-radiation of a gamma and/or X-ray beam, so that an advantageous beam bundling can be achieved and thus an undesired beam spreading which could cause damage to tissue of the patient surrounding the treatment area is prevented. This embodiment of the invention advantageously enables an especially compact embodiment of the apparatus to be achieved. In addition a high radiation dose can be explicitly focused on the treatment area. Especially advantageously the collimator is embodied from magnetic resonance-compatible and from a non-magnetizable material.

In a further embodiment of the invention it is proposed that the main magnet features at least one subarea permeable for the particle beam. In this context a permeable subarea should be understood as especially a subarea transparent for the particle beam which the particle beam passes through and/or penetrates for interaction substantially without the main magnet and/or a magnetic field generated by the main magnet. Through this embodiment of the invention the particle beam can advantageously be introduced into the magnetic resonance apparatus undiverted and an undesired scattering and/or deflection of the particle beam at the main magnet and/or as a result of a magnetic field applied can be prevented. As well as a subarea transparent for the particle beam, the main magnet can, as an alternative or in addition also feature a subarea transparent for a gamma and/or X-ray beam.

In an advantageous embodiment of the invention it is proposed that the main magnet has at least two coaxial magnetic rings for at least two, especially coaxial circular magnetic disks. One direction of the magnetic field can in this case advantageously be aligned in a direction of a first magnetic ring and/or a first circular magnetic disk in the direction of a second magnetic ring and/or a second circular magnetic disk, so that an advantageous positioning of the particle beam and/or of the gamma beam within the magnetic resonance apparatus can be achieved in respect of the patient, especially the irradiation area of the patient. This can be achieved especially advantageously by there being an accommodation area to accommodate a patient arranged between the at least two magnetic rings and/or the at least two circular magnetic disks. The accommodation area in this case includes the examination space. Preferably the patient is arranged within the accommodation area such that a head-foot axis and/or a longitudinal extent of the patient is aligned essentially transverse to the orientation and/or direction of the magnetic field generated by the main magnet.

In addition it is proposed that the transparent subarea is arranged in a center of the magnetic rings and/or of the circular magnetic disks. The particle beam can be introduced in a constructively simple manner into the magnetic resonance apparatus while maintaining a symmetry for the homogeneous magnetic field.

In an alternate development of the invention it is proposed that the apparatus features a patient bed which is arranged within the accommodation area to allow movement in at least two directions. An effective positioning of the patient in respect of an alignment and/or orientation of the particle beam and/or of the gamma and/or X-ray beam of the radiotherapy apparatus can be achieved. Preferably the at least two directions are aligned orthogonally to each other. In addition it is conceivable for the patient bed to be arranged to allow movement in three spatial directions which are preferably aligned orthogonally to each other.

It is further proposed that the main magnet be arranged movably together with at least part of the radiotherapy apparatus in at least one direction. An advantageous positioning of the particle beam and/or of the gamma and/or X-ray beam in respect of the treatment area can be achieved while maintaining the parallelism of the orientation and/or direction of the magnetic flux density of the magnetic field to the orientation and/or direction of the speed of the particle beam. Preferably a gradient system, especially a gradient coil and/or a high-frequency system, especially a high-frequency coil, is moved in the at least one direction together with the main magnet. Especially advantageously the main magnet is also arranged to enable it to be moved with at least part of the radiotherapy apparatus in at least two spatial directions, with the spatial directions preferably being aligned orthogonally to each other.

Furthermore it is proposed that the main magnet be arranged to allow rotation together with at least part of the radiotherapy apparatus around at least one axis. Preferably the axis runs through the accommodation area for the patient so that an efficient irradiation of the patient, especially of the treatment area of the patient, from different angular positions can be achieved. In addition the patient can remain in this case in a position advantageous for them during the radiation treatment.

Especially advantageously the magnetic resonance apparatus is formed by a high-field magnetic resonance apparatus so that a high-quality in the signals of the magnetic resonance measurements recorded can be achieved. Preferably the magnetic field in this case has a magnetic field strength of at least 3 Tesla and advantageously of at least 5 Tesla.

### BRIEF DESCRIPTION OF THE DRAWINGS

Further advantages emerge from the following description of the drawing. The drawing shows exemplary embodiments of the invention. The drawings, the description and the claims contain numerous features in combination. The person skilled in the art would expediently also consider the features individually and group them together into sensible further combinations.

The figures show:

FIG. 1 an inventive apparatus in a schematic representation and

FIG. 2 an alternate embodiment of the inventive apparatus to FIG. 1 in a schematic representation.

### DETAILED DESCRIPTION OF THE INVENTION

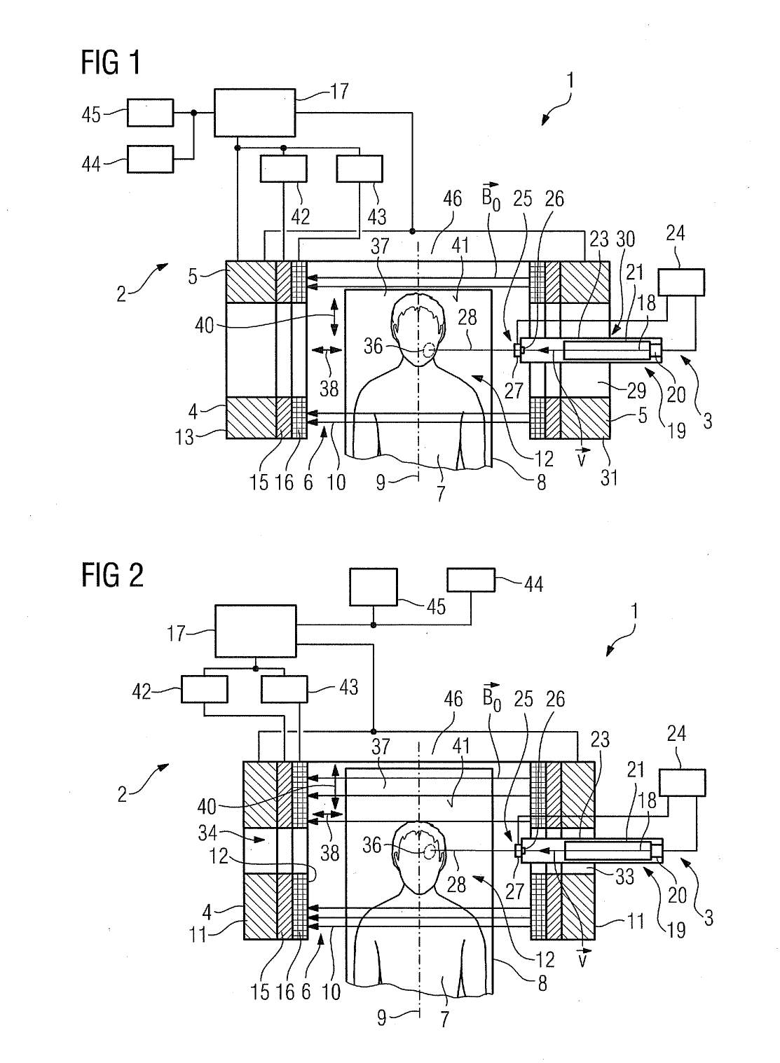

FIG. 1 and FIG. 2 each show a schematic diagram of an inventive apparatus 1 with a combination of a magnetic resonance apparatus 2 and a radiotherapy apparatus 3. The magnetic resonance apparatus 2 comprises a main magnet 4 which is intended, during operation of the magnetic resonance apparatus 2, to generate an especially homogeneous, constant magnetic field with a magnetic flux density \vec{B}₀. The magnetic resonance apparatus 2 is formed by a high-field magnetic resonance apparatus which generates a magnetic field with a high magnetic flux density \vec{B}₀ of at least 3 Tesla and greater during the operation of the magnetic resonance apparatus 2. Basically the magnetic resonance apparatus can also be operated with a magnetic flux density of the magnetic field of less than 3 Tesla, for example with 1.5 Tesla.

The two exemplary embodiments in FIGS. 1 and 2 differ in their design of the main magnet 4 of the magnetic resonance apparatus 2. In FIG. 1 the main magnet 4 is formed by two magnetic rings 5 which are especially formed by supraconducting magnetic rings 5 and which are arranged coaxially to one another. The two coaxial magnetic rings 5 are additionally arranged at a distance from one another, with an accommodation area 6 to accommodate a patient 7 and/or an examination probe being arranged between the two coaxial magnetic rings 5. In the accommodation area 6 the patient 7 is introduced by means of a transport device 8 of the magnetic resonance apparatus 2 in parallel to their longitudinal extent and/or their head-foot axis 9. The transport device 8 is embodied at least partly magnetic-resonance-compatible for this purpose and is formed from a non-magnetizable material.

In FIG. 2 the main magnet 4 is formed by two circular magnetic disks 11. The two circular magnetic disks 11, in a similar manner to the description of an arrangement of the magnetic rings 5 from FIG. 1, are arranged coaxially to one another, with the accommodation area 6 to accommodate the patient 7 likewise being arranged between the two circular magnetic disks 11. By means of the two circular magnetic disks 11 a constant and especially homogeneous magnetic field with a magnetic flux density \vec{B}₀ is generated during operation of the magnetic resonance apparatus 2.

The magnetic field, especially the magnetic flux density \vec{B}₀ acts in the accommodation area 6 for accommodating the patient 7 for magnetic resonance measurement, with the accommodation area including an examination space 12 in which the magnetic resonance measurement is undertaken. An orientation and/or a direction 10 of the magnetic flux density \vec{B}₀ of the magnetic field is embodied in FIG. 1 substantially perpendicular to a ring surface 13 of the two magnetic rings 5. The orientation and/or the direction 10 of the magnetic flux density \vec{B}₀ of the magnetic field runs in this case from a first of the two magnetic rings 5 in a direction towards a second of the two magnetic rings 5. In FIG. 2 an orientation and/or a direction 10 of the magnetic flux density \vec{B}₀ of the magnetic field is in this case embodied substantially perpendicular to a circular disc surface 14 of the two circular magnetic disks 11. The orientation and/or the direction 10 of the magnetic flux density \vec{B}₀ of the magnetic field runs in this case from a first of the two circular magnetic disks 11 in a direction of a second of the two circular magnetic disks 11. In addition the orientation and/or direction 10 of the magnetic flux density \vec{B}₀ of the magnetic field is aligned substantially perpendicular to an alignment of the head-foot axis 9 of a patient 7 located in the accommodation area 6.

A size and/or a number of the magnetic rings 5 included by the main magnet 4 (FIG. 1) and/or by the circular magnetic disks 11 included by the main magnet 4 can in this case be embodied independently of an application area and/or of a type of examination of the magnetic resonance apparatus 2. The size of the magnetic rings and/or of the circular magnetic disks 11 can be dimensioned such that the magnetic resonance apparatus 2 is designed for a whole-body system for a whole-body examination of the patient 7. As an alternative to this the size of the magnetic rings 5 and/or of the circular magnetic disks 11 can have a dimensioning which only makes possible a magnetic resonance examination and/or a magnetic resonance measurement restricted to a limited body area of the patient 7, such as typically, in an application field of neurosurgery, a magnetic resonance examination and/or a magnetic resonance measurement restricted to a head area. In addition the main magnet 4 can also include more than two magnetic rings 5 and/or the circular magnetic disks 11, as can especially be of advantage with magnetic resonance apparatuses 2 for a whole-body system.

Furthermore the magnetic resonance apparatus 2 includes a gradient unit 15 for generating magnetic field gradients. The gradient unit 15 includes gradient coils not shown in greater detail which radiate magnetic gradient fields for selective layer excitation and/or for location encoding of magnetic resonance signals in three spatial directions. To excite a polarization which occurs in the magnetic field of the magnetic resonance apparatus 2 generated by the main magnet 4, a high-frequency coil unit 16 is provided. This beams a high-frequency field, especially in the form of an HF pulse, into the patient 7 in order to deflect a magnetization from a position of equilibrium. Magnetic resonance signals can be detected from an examination space 12 by means of the high-frequency coil unit 16 in measurement operation. Furthermore the magnetic resonance apparatus 2 includes a gradient amplifier 42 for controlling the gradient coils and a high-frequency amplifier 43 for controlling the high-frequency coils. To control the gradient amplifier 42 and the high-frequency amplifier 43, the magnetic resonance apparatus 2 includes a control unit 17. The control unit 17 centrally controls the magnetic resonance apparatus 2, such as the automatic execution of a selected imaging gradient echo sequence for example. For entering magnetic resonance parameters the magnetic resonance apparatus 2 includes an input unit 44. Furthermore the magnetic resonance apparatus 2 includes a display unit by means of which the magnetic resonance images can be displayed for example.

The radiotherapy apparatus 3 is intended to generate a particle beam 18 during operation. To this end the radiotherapy apparatus 3 has an accelerator unit which is formed by a linear accelerator unit 19. The linear accelerator unit 19 is embodied as an electron accelerator unit. Basically an alternate embodiment of the accelerator unit to the linear accelerator unit 19 and/or the electron accelerator unit is conceivable, such as a proton accelerator unit for example.

The linear accelerator unit 19 comprises an electron source 20, such as a tungsten cathode for example, which generates free electrons. These electrons are subsequently accelerated and a particle beam 18 formed by an electron beam is generated. To this end the linear accelerator unit 19 features a cavity resonator 21. In the cavity resonator 21 electrical fields are generated by standing magnetic waves. In addition the cavity resonator 21 comprises several cells arranged one after the other, with a length of the individual cells being selected such that the electric field of the standing wave of a cell reverses as soon as an electron enters the following cell. Thus a continuous acceleration of the electrons to an energy of a few MeV is guaranteed. Furthermore the linear accelerator unit 19 includes a vacuum tube 23 within which the cavity resonator 21 is arranged, so that a vacuum 21 is present in the individual cells of the cavity resonator 21. For control and/or regulation of the linear accelerator unit 19 the X-ray therapy apparatus 3 features a control unit 24.

Furthermore the X-ray therapy apparatus 3 includes a target unit 25 which comprises a target 26 and a collimator 27. The target unit 25 is arranged along the orientation and/or direction of a speed \vec{v} of the accelerated electrons of the electron beam after the cavity resonator 21 of the linear accelerator unit 19. In addition the target 26 is arranged within the vacuum tube 23 of the linear accelerator unit 19 so that an undesired deflection and/or scattering of electrons of the electron beam as a result of a collision between the electrons and air molecules before reaching the target 26 is prevented. The accelerated electrons of the electron beam, after passing through the cavity resonator 21, strike the target 26 of the target unit 25. The target 26 is formed by a transmission target, typically by a tungsten sheet. At the target 26 the accelerated electrons are braked, with gamma radiation 28 and/or X-rays being generated in this case. As an alternative to this it is possible for the target 26 to also be arranged outside the vacuum tube 23.

The collimator 27 is arranged along the orientation and/or direction of the speed \vec{v} of the electron beam after the target 26. A parallel gamma and/or X-ray beam 28 is produced from the gamma and/or X-ray radiation by means of the collimator 27. Furthermore by means of the collimator 27, the gamma and/or the X-ray radiation is focused on a treatment area 36 and/or an isocenter, so that a parallel beam path of the gamma and/or X-ray radiation with a small focus can be made available for radiotherapy during operation of the radiotherapy apparatus 3. The collimator 27 is controlled and/or adjusted by the control unit 24 of the radiotherapy apparatus 3. As an alternative to this however, an at least part manual adjustment and/or control of the collimator 27 is conceivable.

Radiotherapy treatment by means of the radiotherapy apparatus 3 is undertaken simultaneously to a magnetic resonance measurement by means of the magnetic resonance apparatus 2 so that the radiotherapy treatment can be effectively adapted to a movement of patient 7 for example. The radiotherapy apparatus 3 is arranged and/or integrated at least partly for this purpose within the magnetic resonance apparatus 2. The target 26 and the collimator 27 are in this case arranged within the magnetic resonance apparatus 2 and especially within the accommodation area 6 with the homogeneous magnetic field. For this purpose the target unit 25 and at least partly the linear accelerator unit 19 are embodied as magnetic-resonance compatible and are made of a non-magnetizable material.

If electrons and/or charged particles move in a magnetic field, a force \vec{F} acts on the electrons and/or the charged particles which is proportional to a vector product of the speed \vec{v} of the electron beam and the magnetic flux density \vec{B}₀ of the magnetic field acting: \vec{F}=q(\vec{v}×\vec{B}₀). In addition the force \vec{F} is still dependent on a charge q of the electrons and/or the charged particles. In FIG. 1 the orientation and/or direction of the speed \vec{v} of the electron beam is aligned substantially in parallel to the orientation and or direction 10 of the magnetic flux density \vec{B}₀ of the magnetic field, so that an undesired adverse effect of the homogeneous magnetic field and/or an undesired deviation of a particle track of the electron beam is prevented. This means that no forces brought about by the magnetic field of the magnetic resonance apparatus 2 act within the magnetic resonance apparatus 2 on the electrons of the electron beam. In addition the orientation and/or direction of the speed \vec{v} of the electron beam is substantially perpendicular to an alignment of the head-foot axis 9 of the patient 7 located in the accommodation area 6. In addition it is possible for the condition of the parallelism of the magnetic flux density \vec{B}₀ of the magnetic field and the speed \vec{v} of the electron beam to include a tolerance range. The tolerance range has the following condition that the greater a value of the magnetic flux density \vec{B}₀ of the magnetic field is, the more strictly aligned in this case must be the speed \vec{v} of the electron beam in parallel to the direction 10 of the magnetic flux density \vec{B}₀, in order to prevent an undesired deflection of the electron beam.

For an at least part arrangement of the linear accelerator unit 19 within the magnetic resonance apparatus 2, the main magnet features a subarea 29, 33 transparent and/or permeable for the electron beam for this purpose (FIGS. 1 and 2). This subarea 29 transparent and/or permeable for the electron beam is arranged in FIG. 1 in a center 30 of the individual magnetic rings 5 of the main magnet 4. In addition the transparent subarea 29 is enclosed by an annular area 31 of the magnetic rings. In this permeable subarea 29 the electron beam can penetrate substantially unhindered into the magnetic resonance apparatus 2 and especially into the accommodation area 6, without interacting with the magnetic rings 5. The transparent subarea 29 is formed by a cavity.

In FIG. 2 the permeable and/or transparent subarea 33 for the electron beam is arranged within the individual circular magnetic disks 11. For reasons of symmetry this permeable and/or transparent subarea 33 for the electron beam is arranged in a center 34 of the circular magnetic disks 11 and is formed by a cavity.

The linear accelerator unit 19 and/or a beam guidance of the radiotherapy apparatus 3 is arranged within the transparent subarea 29, 33 such that an orientation and/or direction of the speed \vec{v} of the electron beam is aligned substantially in parallel to the magnetic flux density \vec{B}₀ of the magnetic field. The transparent subarea 29, 33 can additionally be formed by a film substantially permeable for the particle radiation. As an alternative to this, further embodiments of the transparent subarea 29, 33 appearing sensible to the person skilled in the art are always possible. The vacuum vessel of the linear accelerator unit 19 extends through this transparent subarea 29, 33, so that in addition to the cavity resonator 21, the target 26 can also be arranged within the vacuum.

As an alternative to this, the linear accelerator unit 19 can also be arranged outside the main magnet 4 and the magnetic field generated by the main magnet 4. In addition it is also conceivable for the target 26 and/or the collimator 27 to be arranged within the transparent subarea 29, 33 of the magnetic rings 5 and/or of the circular magnetic disks 11. Furthermore both the target 26 and also the collimator 27 can also be arranged outside the magnetic field and the main magnet 4. In this case it would be sufficient for the transparent subarea 29, 33 to only be designed transparent for the gamma and/or X-ray radiation.

The electron beam passes through the magnetic resonance apparatus 2 to the target 26 substantially undiverted. An adverse effect from the homogeneous magnetic field which could be caused as a result of a diverted and/or deflected electron beam is thus advantageously suppressed so that a magnetic field with a high magnetic flux density \vec{B}₀ of at least three Tesla can be created during operation. The electron beam is directed even before its entry into the magnetic resonance apparatus 2 onto a treatment area 36, especially an isocenter.

Furthermore the apparatus 1 is designed for carrying out an adjustment of a beam focus of the gamma and/or X-ray beam 28. An adjustment can be necessary if for example the isocenter and/or the treatment area 36 of the radiation treatment moves during the radiation treatment, for example as a result of breathing and/or a movement of the patient 7. Furthermore the isocenter and/or the treatment area 36 can have a greater extent than the extent of the irradiation focus of the gamma and/or X-ray beam 28 so that for a complete irradiation, of a tumor tissue for example, an adjustment of the gamma and or X-ray beam 28 is necessary. The adjustment is carried out on the basis of magnetic resonance images which localize the treatment area 36. For the adjustment of the irradiation focus of the gamma and/or X-ray beam 28 in respect of the movement and/or extension of the isocenter, the apparatus 1 provides two options. On the one hand the transport facility 8 features a patient bed 37 which is arranged to allow movement within the accommodation area 6 in two spatial directions. The two spatial directions are formed by an x direction 38 and a z direction 40 and are each aligned orthogonally to one another. The z direction is additionally aligned in the direction of an insertion process for inserting the patient bed 37 into the accommodation area 7. In addition the patient bed 37 can also be arranged to allow movement in a third spatial direction which is formed by a y direction, with the third spatial direction being aligned orthogonally to the first and the second spatial direction for this purpose.

A further option for adjusting the gamma and/or X-ray beam 28 in respect of the isocenter and/or of the treatment area 36 consists of arranging at least the linear accelerator unit 19 and the target unit 25 of the radiotherapy apparatus 3 to allow movement together with at least the main magnet 4 of the magnetic resonance apparatus 2. The movement of the linear accelerator unit 19 and the target unit 25 of the radiotherapy apparatus must in this case always take place together with at least the main magnet 4 of the magnetic resonance apparatus 2 so that a substantially parallel orientation and/or direction of the speed \vec{v} of the electron beam to the orientation and/or direction 10 of the magnetic flux density \vec{B}₀ of the magnetic field of the main magnet 4 is retained during the movement. To this end the apparatus 1 in FIGS. 1 and 2 features a positioning unit 46 which moves the linear accelerator unit 19 and the target unit 25 of the radiotherapy apparatus 3 together with at least the main magnet 4 of the magnetic resonance apparatus 2 in three spatial directions in relation to the patient 7 and/or the transport apparatus 8. As an alternative to this the movement in only one or two spatial directions can also be possible.

In an alternate embodiment of the apparatus 1 there can additionally be provision that, in addition to the main magnet 4, further units and/or components of the magnetic resonance apparatus 2 are also moved by the positioning unit 46 together with the linear accelerator unit 19 and the target unit 25 of the radiotherapy apparatus 3 in the spatial directions for an adjustment of the gamma and/or X-ray beam 28. In addition further units and/or further components of the radiotherapy apparatus 3 can be moved for adjusting the gamma and/or X-ray beam 28 in the three spatial directions by the positioning unit 46.

The transparent subarea 29, 33 also has an extent which is larger than the extent of a cross-section of the linear accelerator unit 19. In this case, with small positional changes of the irradiation focus a position change of the target unit 25 together with the linear accelerator unit 19 can take place relative to the main magnet 4 of the magnetic resonance apparatus 2. A speed component resulting from the positional change of the electron beam and/or the linear accelerator unit 19 perpendicular to the magnetic flux density \vec{B}₀ can in such cases be ignored because of the small positional change speed.

Furthermore it is also conceivable for the main magnet 4 to have at least two or more subareas 29, 33 transparent and/or permeable for the particle beam 18 and/or the gamma and or X-ray radiation. In particular in this case the linear accelerator unit 19 and/or the particle beam 18 can assume different irradiation positions in relation to the patient 7 and in relation to the main magnet 4 of the magnetic resonance apparatus 2. However in the different irradiation positions an alignment of the particle beam 18 and/or of the linear accelerator unit 19 in respect of a substantially parallel alignment of the speed \vec{v} of the particle beam 18 in relation to the direction 10 and/or orientation of the magnetic flux density \vec{B}₀ of the magnetic field of the main magnet 4 is restricted.

Furthermore the apparatus 1 makes provision for an irradiation of the treatment area 36 from different irradiation angles. The irradiation angle is formed in this case by a three-dimensional spatial angle in relation to a bed surface 41 of the patient bed 37. To this end the linear accelerator unit 19 and the target unit of the radiotherapy apparatus 3 are rotated together with at least the main magnet 4 of the magnetic resonance apparatus 2 around an axis by means of the positioning unit. The axis in this case runs through a center of the accommodation area 6 substantially in parallel to and insertion direction of the patient bed 37 so that the linear accelerator unit 19 and the target unit 25 together with at least the main magnet 4 of the magnetic resonance apparatus 2 can be rotated around the patient 7 and moved into a new irradiation position. The rotation of the linear accelerator unit 19 and the target unit of the radiotherapy apparatus 3 must in such cases always be undertaken together with at least the main magnet of the magnetic resonance apparatus, so that a substantially parallel orientation and/or direction of the speed \vec{v} of the electron beam to the orientation and/or direction 10 of the magnetic flux density \vec{B}₀ of the magnetic field of the main magnet is always maintained during the rotation.

As an alternative to this it is possible for the linear accelerator unit 19 and the target unit 25 of the radiotherapy apparatus 3 and the main magnet 4 of the magnetic resonance apparatus 2 to be in a fixed position and, to vary the irradiation angle, for the patient bed 37 to be tilted together with the patient 7 around an axis.

1.-19. (canceled)

20. A magnetic resonance apparatus, comprising:
a main magnet for generating a magnetic field in an examination area for a magnetic resonance measurement; and
a radiotherapy apparatus for generating a particle beam having a direction of a speed aligned in parallel to a direction of a magnetic flux density of the magnetic field.

21. The apparatus as claimed in claim 20, wherein the particle beam runs at least partly within an area through which the magnetic field passes within the magnetic resonance apparatus.

22. The apparatus as claimed in claim 20, wherein the particle beam runs substantially undiverted

23. The apparatus as claimed in claim 20, wherein the particle beam is directed onto a treatment area before entering an area penetrated by the magnetic field of the magnetic resonance apparatus.

24. The apparatus as claimed in claim 20, wherein the direction of the magnetic flux density of the magnetic field is substantially perpendicular to an alignment of a head-foot axis of a patient located in the examination area.

25. The apparatus as claimed in claim 20, wherein the radiotherapy apparatus comprises a linear accelerator unit that is arranged at least partly within the magnetic resonance apparatus.

26. The apparatus as claimed in claim 20, wherein the radiotherapy apparatus comprises a target for generating a gamma and/or X-ray beam and the target is arranged at least partly within the magnetic resonance apparatus.

27. The apparatus as claimed in claim 26, wherein the target is arranged within an area through which the magnetic field passes.

28. The apparatus as claimed in claim 20, wherein the target is at least partly a transmission target.

29. The apparatus as claimed in claim 20, wherein the radiotherapy apparatus comprises a collimator that is arranged at least partly within the magnetic resonance apparatus.

30. The apparatus as claimed in claim 20, wherein the main magnet comprises a subarea permeable for the particle beam.

31. The apparatus as claimed in claim 30,
wherein the main magnet comprises at least two coaxial magnetic rings, and
wherein the subarea is arranged in a center of the at least two coaxial magnetic rings.

32. The apparatus as claimed in claim 31, wherein an accommodation area to accommodate a patient is arranged between the least two coaxial magnetic rings.

33. The apparatus as claimed in claim 30,
wherein the main magnet comprises at least two circular magnetic disks, and
wherein the subarea is arranged in a center of the at least two circular magnetic disks.

34. The apparatus as claimed in claim 33, wherein an accommodation area to accommodate a patient is arranged between the least two circular magnetic disks.

35. The apparatus as claimed in claim 20, wherein a patient bed is arranged within an accommodation area that is moved in at least two directions.

36. The apparatus as claimed in claim 20, wherein the main magnet is moved together with at least part of the radiotherapy apparatus in at least one direction.

37. The apparatus as claimed in claim 20, wherein the main magnet is rotated together with at least part of the radiotherapy apparatus around at least one axis.

38. The apparatus as claimed in claim 20, wherein the magnetic resonance apparatus is a high-field magnetic resonance apparatus.
